(19) United States
(12) Patent Application Publication
(10) Pub. No.: US 2005/0049232 A1
(43) Pub. Date: Mar. 3, 2005

(54) Composition

(76) Inventor: Par Lindau, Andersvagen (SE)

Correspondence Address: YOUNG & THOMPSON, 745 SOUTH 23RD STREET, 2ND FLOOR, ARLINGTON, VA, 22202, US

(21) Appl. No.: 10/499,148

(22) PCT Filed: Dec. 20, 2002
(86) PCT No.: PCT/SE02/02421

(30) Foreign Application Priority Data
Dec. 21, 2001 (SE) 0104421-3

(51) Int. Cl. A61K 7/06; A61K 31/56; A61K 31/505

(52) U.S. Cl. 514/171; 514/275; 424/70.1

(57) ABSTRACT

A pharmaceutical and/or cosmetical composition includes one or more substances active in preventing hair loss and/or stimulating hair growth and a preparation with lipophilic and hydrophilic components for application on the skin, which preparation exists as a two-phase system and thereby is capable of creating a semi-permeable membrane in the skin. The process for the manufacture of the skin preparation as defined above includes dissolving the lipophilic components in water in a separate container to be combined with hydrophilic components, which have been blended and brought to react in another vessel.

The present invention relates to a composition for preventing hair loss and/or stimulating hair growth and a process for the preparation thereof.

### BACKGROUND OF THE INVENTION.

In recent years a number of compounds for the treatment of hair loss have been developed. Hair loss treatment with such compounds is generally provided by delivery of the active ingredient or ingredients through the skin, i.e. transdermally, as opposed to other methods of parenteral administration. The large surface area of the skin and the extensive circulatory and lymphatic networks available near the skin surface make topical application of drugs for treatment of hair loss extremely desirable. Moreover, topical application of drugs is relatively non-invasive, convenient, proven to be safe and provides greater control over delivery of active agents to the desired target site.

However, in order to be effective, an active agent for hair treatment must pass through the outer layer of skin or epidermis and into the dermis before reaching hair follicles and being absorbed into the bloodstream. The epidermis comprises two main parts, the stratum corneum and the stratum germinativum. The stratum corneum forms the outermost layer of the epidermis and consists of many stratified layers of compacted, flattened, keratinized cells, which have lost their nuclei. This outermost layer serves as a physical barrier to microorganisms and most chemical agents and also controls water loss from the body. In particular, it behaves as a primary barrier to percutaneous absorption of drugs. Because of the barrier effect of the skin, it has heretofore only been possible to deliver drugs that are “low-dose” drugs, i.e. in the range of 15 mg/day or less and has a low molecular weight. In addition, drugs for transdermal delivery must have the proper lipophilic-hydrophilic balance to permit adequate absorption. As early as the beginning of this century it has been known that lipid-soluble substances, such as non-electrolytes have a comparatively greater skin permeability than water-soluble substances, such as electrolytes.

Percutaneous absorption or transdermal permeation is basically a composite of a series of steps in sequence. A penetrant molecule is first absorbed onto the surface layers of the stratum corneum, diffuses through the stratum corneum and the viable epidermis below, and finally diffuses through the papillary dermis and into the microcirculation.

Diffusional resistance of the stratum corneum to topically applied agents has been demonstrated with various drugs. In order to overcome this barrier effect a number of compounds have been developed which enhance the transdermal delivery of drugs, such as dimethyl sulfoxide (DMSO), polyethylene glycol monolaurate, alkyl lactams, long chain amides, and substituted 1,3-dioxacyclopentanes and substituted 1,3-dioxacyclohexanes. For example, U.S. Pat. No. 3,551,554 discloses DMSO, U.S. Pat. No. 3,989,816 discloses 1-substituted azacycloheptane-2-one, U.S. Pat. No. 4,132,781 discloses a topical antibiotic composition containing 2-pyrrolidone or an n-lower alkyl-2-pyrrolidone, U.S. Pat. No. 4,017,641 discloses propylene glycol and 2-pyrrolidone-containing compositions and U.S. Pat. No. 4,861,764 discloses 1,3-dioxolane and 1,3-dioxane derivatives as percutaneous absorption enhancers. WO 92/16236 discloses methods and compositions for enhancing the rate of absorption of topically administered physiologically active compounds. Minoxidil is disclosed as one of these compounds, which is used in Rogain® and Regain®. The penetration enhancers are amino alcohol derivatives, which may form a 1,3-dioxane ring.

Therapy of hair loss in androgenic alopecia patients with topical solutions of minoxidil (Rogaine.RTM.) alone, or in combination with skin penetration enhancers, such as DMSO, has resulted in only moderate to dense regrowth of hair in less than 40% of such patients (Katz, H. I., Clin. Dermatol., 6:195-199, 1988). Moreover, treatments with topical solutions of minoxidil require multiple applications of the active ingredient each day, which can be very inconvenient as well as expensive. Androgenic alopecia or common baldness represents 99 percent of all cases of hair loss (Brodland and Muller, Cutis, 47:173-176, 1991) and, therefore, there is a need for improved compositions and methods for treating hair loss in patients with age related baldness. In particular, there is a need for methods of treating hair loss that require fewer applications of active ingredient, e.g. minoxidil, and which will also provide hair regrowth sooner, in more abundance, and thicker, than is presently observed using minoxidil and known penetration enhancers.

Minoxidil is a peripheral vasodilator used in the treatment of hypertension and as a topical solution for the treatment of androgenic alopecia (available as Rogain® and Regaine® 2% and 5% in 85 and 32 countries respectively). Minoxidil exhibits a low systemic absorption following topical administration and the minoxidil serum concentrations curves are characteristically flat for the duration of a 12-hour dosing interval. However, there is a certain systemic effect such as lowering of blood pressure, liquid retention, tachycardi, angina pectoris, pericardia, and hart tamponade.

It is, therefore, an object of the present invention to provide a treatment for hair loss that is safe, with less systemic effects, less topical side effects and simple to use, easy to apply, when compared to other hair loss treatments.

It is also an object of the present invention to provide a treatment for hair loss that provides faster and more abundant hair regrowth than conventional treatments, using fewer applications of the active substance and using the same concentration thereof per treatment as is conventionally used.

### SUMMMARY

It has now turned out that a composition comprising a substances active in preventing hair loss and a preparation comprising lipophilic and hydrophilic components existing as a two-phase system and capable of creating a semi-permeable membrane in the skin, may obviate the above mentioned drawbacks.

The active substance stays and is activated in the right place in the skin. There are fewer side effects in the form of skin reactions, such as skin irritation and less systemic effects. Thus, the concentration of the active substance in the blood is lower as is the lowering of blood pressure. Further a lower dose may be used than for Menoxidil and still the hair growth effect is the same. The composition according to the invention may be used by any human individe irrespective of sex and race. One advantage is that alcohol need not to be used. Alcohol may be a drawback for certain ethnical groups or genotypes.

Therefore the present invention relates to a composition for topical application to the scalp for promoting hair regrowth and a process for preparing thereof.

It also relates to an improved method for treating hair loss, the improvement being topically applying the composition once per day to skin at a desired area for hair regrowth.

### DETAILED DESCRIPTION OF THE INVENTION.

One object of the invention is a pharmaceutical and/or cosmetical composition, which comprises one or more substances active in preventing hair loss and/or stimulating hair growth and a preparation, which comprises lipophilic and hydrophilic components for application on the skin. The preparation exists as a two-phase system and thereby is capable of creating an osmotic, semi-permeable membrane in the skin and is described in WO 98/31339. Thanks to the two phases the skin preparation will act simultaneously on several levels in the skin.

The active substance may be any substance that prevents hair loss, such as alopecia, or stimulates hair growth. The substances may be chosen from pyrimidine and pyrimidine derivatives and from piperidino-pyrimidines and derivatives thereof. Such substances may be 2-amino-pyrimidine-3-oxide; 2,4-diamino-pyrimidine-3-oxide; 6-substituted 2.4-diamino 6-alkoxy-pyrimidine-3-oxides; such as 2.4-diamino 6-alkoxy-or 6-thioalkyl-pyrimidine-3-oxides; 2-alkyl-4-amino (or 2,4-dialkyl or 2.4-diamino)-pyrimidine-3-oxides; 6-haloalkoxy-pyrimidine-3-oxides; 2,4,6-triamino-pyrimidine-3-oxide; and derivatives thereof described, for example, in EP-0,353,123, EP-0,356,271, U.S. Pat. No. 5,466,694, EP-0,408,442, EP-0,522,964, EP-0,420,707, EP-0,459,890 and EP-0,519,819; and especially 2,4-diamino-6-piperidino-pyrimidine-3-oxide; “Minoxidil” described in U.S. Pat. Nos. 3,461,461, 4,139,619 and 4,596,812 and derivatives thereof.

Also 17β-N-(monosubstituted)-carbamoyl-4-aza-5.alpha.-androsten-1-en-3 one compounds described in U.S. Pat. No. 4,760,071, may be used especially 17β-(N-tert-butyl carbamoyl)-4-aza-5 alpha-androst-1-en-3-one, Finasterid.

Derivatives of 2-amino-pyrimidine-3-oxide may be selected from compounds with the formula I: 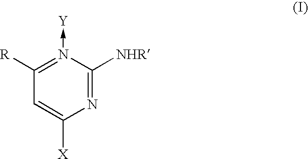
in which:
R denoes a hydrogen, a saturated, straight C1-C8 alkyl radical or R denotes together with the pyrimidine ring, a hydrocarbon ring of formula: 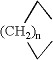
n is an integer of 1, 2 or 3;
X is:
   (i) a hydrogen atom;
   (ii) a group 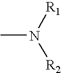
      in which:
R1 and R2, are the same or different and, designate a hydrogen atom, a saturated, straight or branched C1-C12 alkyl group, which may be substituted by a halogen atom or a trifluoromethyl group, a straight C2-C12 alkenyl group, a C3-C10 cycloalkyl group, a C7-C12 aralkyl group or an aryl group with the formula 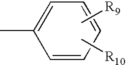
   in which R9 and R10 are same or different and designate hydrogen, C1-C4 alkyl, hydroxyl, C1-C4 alkoxy or halogen;
R1 and R2 may, together with the nitrogen atom to which they are bound, form a saturated or unsaturated heterocycle, chosen among the following groups aziridino, azetidino, pyrrolidino, piperidino, hexamethyleneimino, heptamethyleneimino, octamethyleneimino, tetrahydropyridino, dihydropyridino, pyrrole, pyrrazole, imidazole, triazole, alkyl-4 piperazino, morpholino, thiomorpholino;
   (iii) a group —OR3, in which R3 designates a straight or branched saturated C1-C12 alkyl, which may be substituted by a halogen atom or a trifluoromethyl group, a straight C2-C12 alcenyl, a C3-C10 cycloalkyl group, a C7-C12 aralkyl group, a phenyl group which may be substituted one or two groups which, independently designate a C1-C6 alkyl group, a C1-C6 alkoxy group, a halogen atom or a trifluoromethyl group;
   (iv) a group —SR4, in which R4 has the same meaning as R3 defined above;
Y is an oxygen atom or a group OSO₃⁻;
R1 is a hydrogen atom or one of the following groups: 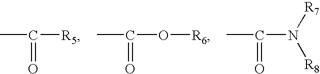
   in which:
R5 and R6 represent lower C1-C4 alkyl groups;
R7 and R8 represent a hydrogen atom or a lower C1-C4 alkyl group, with the provision that they do not simultaneously designate a hydrogen atom.

When Y is un an oxygen atom, the compounds with formula (I), may co-exist with their tautomere with formula (I) according to the following equillibrium:

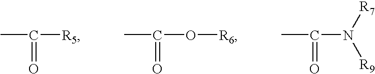

The copounds may especially be chosen among:
-2-amino-4 methyl-6-(2,3-dimethyl)-propyloxy-pyrimidine-3-oxide;
-2-amino-4 methyl-6-(1-cyclohexyl)-methyloxy-pyrimidine-3-oxide;
-2-amino-4 methyl-6-(5-hexenyl)-oxy-pyrimidine-3-oxide;
-2-amino-4 methyl-6-benzyloxy-6 pyrimidine-3-oxide;
-2-amino-4 methyl-6-(2,4-dimethyl)-phenyloxy-pyrimidine-3-oxide;
-2-amino-4 methyl-6-methylthio-pyrimidine-3oxide;
-2-amino-4 methyl-6-ethylthio-pyrimidine-3-oxide;
-the inner salt of 2-amino-4 methyl-6-methoxy-3-sulfoxy-pyrimidinium-hydroxide;
-the inner salt of 2-amino-4 methyl-6-ethoxy-3-sulfoxy-pyrimidinium-hydroxide;
-2-amino-4,5-tetramethylene-6-butoxy-pyrimidine-3-oxide;
-the inner salt of 2-amino-4,5-tetramethylene-6-butoxy-3-sulfoxy-pyrimidinium-hydroxide.

The active compounds in accordance with the present invention can also be 2,4-diaminopyrimidine 3-oxides of formula II: 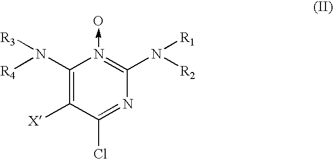
in which: R1 and R3 denote a hydrogen and R2 and R4 denote a hydrogen or a C1-C4 alkyl radical and X′ denotes a hydrogen or a C1-C6 alkyl radical or a group —NHR6 where R6 denotes an alkyl radical or a C1-C6 acyl radical.

2.4-diamino-6-alkoxy-pyrimidine-3-oxides; and 2.4-diamino-6-thioalkyl-pyrimidine-3-oxides may also be used.

These may be compounds corresponding to the formula III: 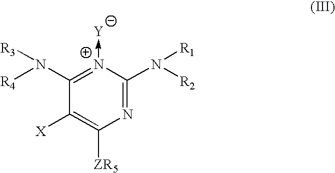
in which: R1 and R3 denote a hydrogen atom;
R2 and R4, which are identical or different, represent a hydrogen atom or a C1-C4 alkyl group;
R5 denotes a hydrogen atom, a C1-C12 alkyl radical, a C3-C12 alkenyl radical, a C3-C8 cycloalkyl radical, an aryl radical, an arylalkyl radical or a hydroxyalkyl or aminoalkyl radical in which the alkyl radical has 1 to 6 carbon atoms;
X denotes a hydrogen atom, a halogen atom, a C1-C6 alkyl radical, a nitro group, a benzoyloxy group or a group —NHR6 in which R6 denotes a hydrogen atom, an acyl radical or a C1-C6 alkyl radical;
Z denotes sulfur or oxygen, with the proviso that Z denotes sulfur when X denotes hydrogen or when R5 denotes an aryl radical;
Y denotes oxygen or OSO₃⁻;
as well as its addition salts with physiologically acceptable acids.

The halogen atoms in the structure of formula (III) are preferably chlorine, bromine, fluorine or iodine.

The C1-C6 alkyl radicals are preferably chosen from methyl, ethyl, propyl, n-butyl, n-pentyl and n-hexyl.

The C1-C12 alkyl radicals are preferably chosen from methyl, ethyl, propyl, 2-ethylhexyl, decyl, octyl and dodecyl.

The C3-C12 alkenyl groups are preferably chosen from allyl, n-butenyl, hexenyl and dodecenyl.

The aryl or aralkyl radicals are preferably chosen from phenyl, benzyl and tolyl.

In preffered embodiments the C1-C6 alkyl radicals are methyl, ethyl, propyl, n-butyl, n-pentyl or n-hexyl; the C1-C12 alkyl radicals are methyl, ethyl, propyl, n-butyl, n-pentyl, n-hexyl, 2-ethylhexyl, octyl, decyl or are dodecyl; the C3-C12 alkenyl groups are allyl, butenyl, hexenyl, decenyl or dodecenyl; the aryl radicals are phenyl or benzyl; and the halogen atoms are chlorine, bromine, fluorine or iodine.

Among the compounds of the invention of general formula (III) in which Z denotes oxygen, the particularly preferred compounds are those for which R1, R2, R3 and R4 denote hydrogen, X denotes chlorine or the nitro group and R5 denotes n-butyl.

Among the compounds of the invention of formula (III) in which Z denotes sulfur, the particularly preferred compounds are those for which X denotes chlorine or the nitro group, R1, R2, R3 and R4 denote a hydrogen atom and R5 denotes methyl, ethyl, n-butyl, 2-hydroxyethyl, 2-aminoethyl or phenyl.

Preffered compounds are those, wherein in the compound of formula (III) Z denotes sulfur; X denotes chlorine or nitro; R1, R2, R3 and R4 denote hydrogen and R5 denotes methyl, ethyl, n-butyl, phenyl, 2-hydroxyethyl or 2-aminoethyl: and those in which the compound of formula (III) is 2,4-diamino-5-chloro-6-n-butyloxypyrimidine 3-oxide or 2,4-diamino-5-nitro-6-n-butyloxypyrimidine 3-oxide.

Preffered compounds are further those in which Y is oxygen and R1, R2, R3, R4, Z and R5 have the same meanings indicated above in the general formula (III), and X denotes hydrogen, a C1-C6 alkyl radical or a group —NHR6 where R6 denotes a C1-C6 radical or an acyl radical.

Other useful 6-substituted 2,4-diamino-pyrimidine-3-oxides that may be used are compounds of the formula IV: 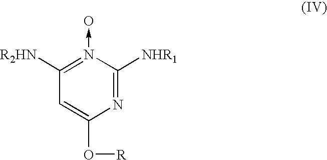
in which: R1 and R2 denote, independently of each other, a hydrogen atom, a carbamoyl group of formula: 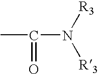
with R′3=H or R3; or an alkoxycarbonyl group of formula: or an acyl group of formula: 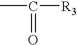
in which formulae R3 denotes a linear or branched C1-C18 alkyl radical, a C2-C18 alkenyl group or a C5-C8 cycloalkyl group; R3 may also denote an aryl or aralkyl radical corresponding to the formula: 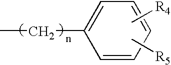
in which: n is an integer which can vary between 0 and 4;
R4 and/or R5, independently of each other, denote hydrogen, a lower C1-C6 alkyl group, a hydroxyl or C1-C6 alkoxy group, a halogen atom or a CF3 group;
R denotes a linear or branched C1-C18 alkyl or C2-C18 alkenyl radical, a C4-C6 cycloalkyl radical capable of bearing an unsaturation or a C1-C6 alkyl radical substituted with phenyl or pyridine.

The compounds in accordance with the invention which are more particularly preferred are the compounds in which the alkyl group denotes, unless indicated otherwise, a group containing 2 to 12 carbon atoms and the aromatic nucleus preferably denotes phenyl.

The particularly preferred compounds defined above are those in which R is chosen from methyl, ethyl, butyl, isobutyl, n-hexyl, n-octyl, n-decyl, lauryl, 5-n-hexenyl, 2-ethylhexyl, 10-undecenyl, cyclohexyl, phenethyl or benzyl groups.

The particularly preferred compounds of the invention consist of 2,4-diamino-6-(n-butyloxy)pyrimidine 3-oxide, 2,4-diamino-6-ethyloxypyrimidine 3-oxide, 2,4-diamino-6-methoxypyrimidine 3-oxide, 2,4-diamino-6-n-hexyloxypyrimidine 3-oxide,2,4-diamino-6-n-octyloxypyrimidine 3-oxide, 2,4-diamino-6-n-dodecyloxypyrimidine 3-oxide, 2,4-diamino-6-(2-ethylhexyloxy)pyrimidine 3-oxide, 2,4-diamino-6-(5-n-hexenyloxy)pyrimidine 3-oxide, 2,4-diamino-6-(10-undecenyloxy)pyrimidine 3-oxide, 2,4-diamino-6-(2-phenylethyloxy)pyrimidine 3-oxide, 2,4-diamino-6-(2-trifluoroethyloxy)pyrimidine 3-oxide, 2-amino-4-acetamido-6-butyloxypyrimidine 3-oxide, 2,4-di(benzyloxycarbonylamino)-6-butyloxypyrimidine 3-oxide, 2,4-di(methoxycarbonylamino)-6-butyloxypyrimidine3-oxide, 2,4-di(benzyloxycarbonylamino)-6-butyloxypyrimidine 3-oxide and N-(2-amino-6-butyloxy-4-pyrimidinyl)-N′-dimethyl-urea 3-oxide, 2,4-diaminopyrimidine 3-oxides, chosen from 2,4-diamino-6-methoxypyrimidine 3-oxide, 2,4-diamino-6-n-hexyloxypyrimidine 3-oxide, 2,4-diamino-6-n-octyloxypyrimidine 3-oxide, 2,4-diamino-6-n-dodecyloxypyrimidine 3-oxide, 2,4-diamino-6-(2-ethylhexyloxy)pyrimidine 3-oxide, 2,4-diamino-6-(5-n-hexenyloxy)pyrimidine 3-oxide, 2,4-diamino-6-(10-undecenyloxy)pyrimidine 3-oxide, 2,4-diamino-6-(2-phenylethyloxy)pyrimidine 3-oxide, or cosmetically or pharmaceutically acceptable salts.

The compounds of formula IV can also exist in their tautomeric form, corresponding to the following formulae (IVA) and (IVB):

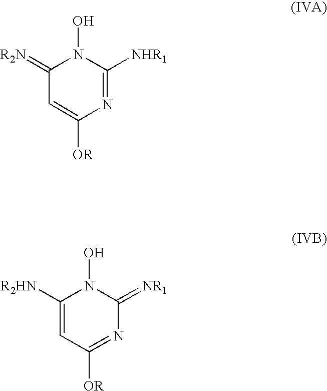

These tautomeric forms (IV), (IVA) and (IVB) can be present in variable proportions and one can be predominant in relation to the others. 2-alkyl-4-amino (or 2,4-dialkyl or 2.4-diamino)-pyrimidine-3-oxides of formula V, may also be used: 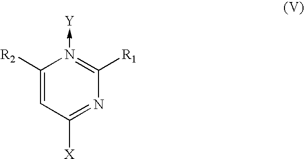
in which:
R1 is a hydrogen atom or a straight saturated C1-C8 alkyl group;
R2 is a straight saturated C1-C8 alkyl group a group —NRH3 in which R3 is a hydrogen atom or a groupe —COOR4 where R4 is a straight C1-C4 alkyl group;
X is:
   i) a group 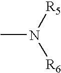
      is which:
R5 and R6, which are identical or different represent a hydrogen atom, a straight or branched C1-C12 alkyl group, which may be substitted by one or more halogen atoms, a straight C2-C12 alcenyl group, a C3-C10 cycloalkyl group or an aryle group or an aralkyl group with the formula: 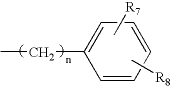
   wherein
n is 0-4;
R7 and/or R8, indipendently designate a hydrogen atom, a lower C1-C6 alkyl or C1-C6 alkoxy group or a trifluoromethyl group;
R5 andt R6 may form together with the nitrogen atom to which they are linked a saturated or insaturated heterocyclic group chosen from the following groups: aziridino, azetidino, pyrrolidino, piperidino, hexamethyleneimino, heptamethyleneimino, octamethyleneimino, tetrahydropyridino, dihydropyridino, pyrrole, pyrrazole, imidazole, triazole, alkyl-4 piperazino, morpholino, thiamorpholino;
   (ii) a group —OR9, in which is a straight or branched C1-C12 alkyl group, which may be substitued by one or more halogen atoms, un a C1-C12 alcenyl group, a C3-C10 cycloalkyl group, a C7-C12 aralkyl group, a phenyl group which may be substitued by one or more groups which indepentently designate a C1-C6 alkyl group, a C1-C6 alkoxy group, a halogen atom of a trifluoromethyle group;
   (iii) a groupt-SR10, in which R10 has the same meaning as R9 defined above;
Y is a oxygen atom or a group —OSO₃⁻.

When in formula V, X is a group 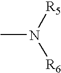
the preferred compounsd are those in which:
R2 is a methyl group and X is piperidino; or
R2 is a group —NHR3, in which R3 has the same meaning as in formula V, and X is one of the following amino groups: dimethylamino, diethylamino, n-butylamino, piperidino, morpholino, methyl-4 piperazino, benzylamino or anilino.

When in formula V, X is a group —OR9, the preferred compounsd are those in which:
R2 is a methyl group and X is a ethoxy group; or
R2 is a group —NHR3, in which R3 has the same meaning as in formula (V) and X designates the following alkoxy groups: ethoxy, butoxy, 1-methyl ethyloxy and 2,4-dimethyl-phenyloxy.

When Y is an oxygen atom and R2 is a group —NHR3, the compounds of formula (V) exist in two tautomeric forms, according to the equilibrium below:

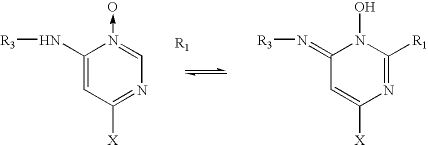

Preferred compounds are:
2-methyl-4 amino-6-ethoxy-pyrimidine-3-oxide;
2-methyl-4 amino-6-(2,4-dimethyl-phenyl)-oxy-pyrimidine-3-oxide;
2-methyl-4 amino-6-(1-methyl)-ethyloxy-pyrimidine-oxide;
2-methyl-4 amino-6-butoxy-pyrimidine-3-oxide;
2-methyl-4-ethoxycarbonylamino-6-piperidino-pyrimidine-3-oxide;
2,4-dimethyl-6-piperidino-pyrimidine-3-oxide;
the inner salt of 2,4-dimethyl-6-piperidino-3-sulfoxy-pyrimidinium-hydroxyde.

6-haloalkoxy-pyrimidine-3-oxides with the following formula VI may also be used: 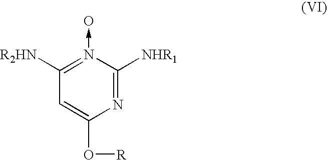
in which:
R1 and R2 independently designate an hydrogen atom, a carbamoyl group of the formula: 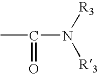
   in which R′3 designates hydrogen or R3;
a alkoxycarbonyl of the formula: 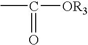
   or a acyle groupde of theformula: 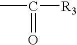
   in which R3 designates a straight or branched C1-C18 alkyl group, a C2-C18 alkenyl group, an C5-C8 cyclo alkyl group; or R3 designates a aryl or aralkyl group of the formula: 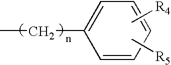
   in which:
n is a number 0-4;
R4 and/or R5, represent independently hydrogen, a lower C1-C6 alkyl group, a hydroxyl group, hydroxyl, a C1-C6 alkoxy group;
R is a straight or branched C1-C6 alkyl group, substituted by one or more halogen atoms;
and cosmetcially and pharmaceutically acceptable acid addition salts.

Preferred coupounds are those in which the halogen atoms are fluor or chlor; the C1-C6 alkyl group which R may especially represent is, a mono- or polyhalo methyl, -ethyl or -propyl group; and R1 and R2 preferably designate hydrogen.

Preferred coupounds are those in which the halogen atoms are fluor or chlor; the C1-C6 alkyl group which R may especially represent is, a mono- or polyhalo methyl, -ethyl or -propyl group; and R1 and R2 preferably designate hydrogen.

Preferred R groups are chosen from —CH2CF3, —CH2—CF2—CHF2.

Preferred compounds are 2,4-diamino-6-(2-trifluoro-ethyloxy)-pyrimidine-3 oxide and 2,4-diamino-6-(2,2,3,3-tetrafluoro-propyloxy)-pyrimidine-3-oxide.

These compounds may exist in tautomic forms with formual (VIA) and (VIB) below:

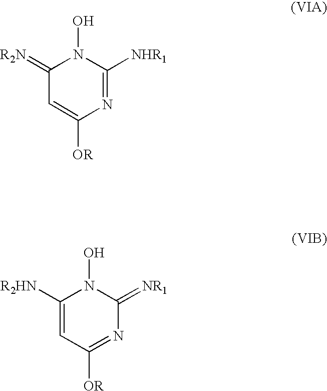

The tautomeric forms (VI), (VIA), and (VIB) may be present in different proportions.

Compounds my also be chosen from mono-or dicarbamides in the 2-and 4-positions of 2,4,6-triamino-pyrimidine-3-oxide in which the nitrogen atom of the carbamide function which is not bound to the pyrimidine ring is substituted or disubstituted by a group having one or more hydroxyl or groups or when it is disubstituted forms a heterocyclic ring having one or more heteroatom(s).

These substances may have the following formula VII: 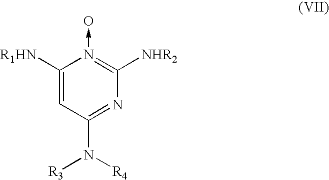
in which:
R3 and R4, which may be identical or different, designate a dhydrogen atom, a straight or branched C1-C18 alkyl group, C2-C18 alkenyl, C5-C8 cycloalkyl which may be substitutedby one or more lower alkyl group(s), a cycloalkene group, whereby the alkyl, alkenyl or cycloalkyl groups in their turn may be substituted by one or more hydroxyl group(s),
R3 and/or R4 designate also a aryl group or a aralkyl group, with the formula: 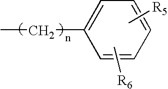
   in which:
n is a number 0-4 and R5 and/or R6, represent independently hydrogen, a lower C1-C6 alkyl group, a nitro, hydroxyl, alkoxy group or a halogen atom, or a carboxyl group and their salt, ester amd amides,
R3 and R4, may togehter with the nitrogen atom to which they are bound form a heterocycle with 3-7 carbon atoms,
R1 and/or R2, designate independently a hydrogen atom or a carbamoyle group of the formula: 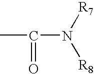
   with the provision that R1 and R2 may not represent a hydrogen atom at the same time, and
   in which:
R7 and/or R8 independently designate straight or branched C1-C18 alkyl, substituted by one or more hydroxyl and/or amino group(s), which alkyl chain may be interrupted by one or more heteroatome(s) such as a sulphur, notrogen or oxygen atom, a cycloalkyl, aryl or aralkyl group, substituted by a hydroxyl, amino and/or carboxylic group; R7 and/or R8 may also designate a sugar rest; R7 and R8 may form together with the nitrogen atom to which they are bound a heterocyclidc ring having one or more heteroatom(s).

The compounds may also exist in the tautomeric form according to the formulas (VIIA) and (VIIB):

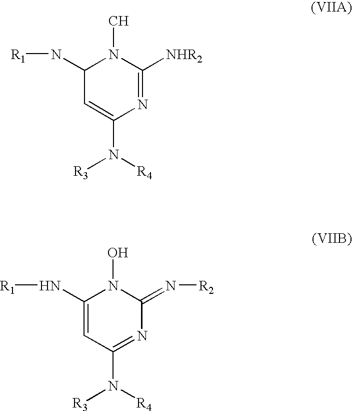

Depending on the substitutents R3, R4, R1, R2 or the nature of the solvant, the tautomeric formes (VII), (VIIA) and (VIIB) may be presented int the mixture in different proportions.

The C1-C18 alkyl groups substituted by one or more hydroxyl(s) or amino group(s) are preferably chosen from the 2-hydroxy-ethyle, 2,3-dihydroxy-propyl, 2-hydroxy-propyl, 2-hydroxy-ethoxyethyl, 2-hydroxy-ethylaminoethyl, 2-amino-ethyl groups.

The cycloalkyl, substituted by one or more hydroxyl(s) or amino group(s) are preferably C5-C8 cycloalkyl groups. When R7 and R8 together with the nitrogen atom to which they are bound form a heterocyclic ring having one or more heteroatom(s), preferred groups are morpholino, piperidino, pyrrolidino, piperazino, N-alkyl4′-piperazino, in which the alkyl in 4′-position preferably contains 1-6 carbon atoms, of which one may be substituted by a hydroxyl group or corresponds to a thiazolidine.

Preferred compounds are those in which one of the groups R1 or R2 designate a hydrogen atom and the other group R2 or R1 does not represent hydrogen and having the following formula (VIIC): 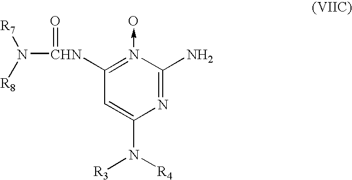
in which R3, R4, R7 and R8 have the same meaning as in formula (VII) above.

Preferred compounds are also those with the formula (VIID) and their isomers (VIIE): 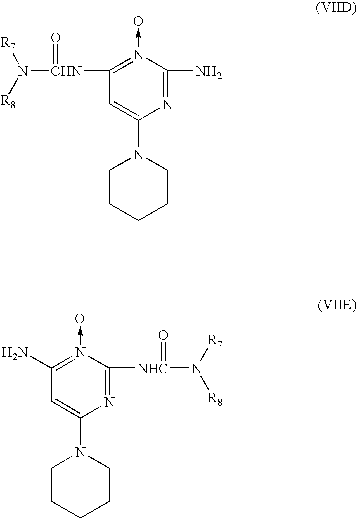
in which R7 and R8 have the same meanings as above.

Other N-oxide pyrimidines substituted in the 2,4 and 6-position that may be used according to the invention are substances of the formula VIII. 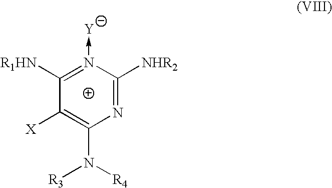
in which:
R1 and R2, independently designate a hydrogen atom or a C1-C8 alkyl group, with the provision that they do not simultaneously represent a hydrogen atom;
R3 and R4, independently designate a hydrogen atom or a C1-C8 alkyl group or form together with the nitrogen atom bound in the 6-position of the pyrimidine ring a
C3-C8 heterocyclic ring, with the provision that they do not simultaneously represent a hydrogen atom;
X is a hydrogen or halogen atom;
Y is O or OSO₃⁻.

Preferred C1-C8 alkyl groups are methyl, ethyl, n-propyl, isopropyl, n-butyl, tertiobutyl, octyl, 2-ethyl-hexyl, hexyl.

Preferred halogen atoms are chlore or brome.

A heterocyclic groups with C3-C8, are preferably morpholino, piperidino, pyrrolidino, piperazino, N-alkyl-4′ piperazino, in which the alkyl group in 4′-position preferably contains 1 to 6 carbon atoms.

Preferred compound are 2-amino 4-propylamino 6-dimethylamino-pyrimidine-3-oxide, 2-amino-4-isopropylamino-6-dimethylamino-pyrimidine-3-oxide, 2,4-N,N-dipropylamino-6-dimethylamino-pyrimidine-3-oxide, 2-propylamino-4-amino-6-N,N-dimethylamino-6-pyrimidine-3-oxide, 2-amino 4-methylamino-6-pyrrolidino pyrimidine-3-oxide, 2-amino-4-tertiobutylamino-6-dimethylamino-pyrimidine-3-oxede, 2,4-N,N-diethylamino-6-dimethylamino-pyrimidine-3-oxide, 2-amino-4-propylamino-6-piperidino-pyrimidine-3-oxide, 2-amino-4-butylamino-6-diethylamino-pyrimidine-3-oxide, 2-amino-4-ethylamino-6-dimethylamino-pyrimidine-3-oxyde, 2-amino-4-propylamino-5-chloro 6-dimethylamino-pyrimidine 3-oxide.

6-Amino-4-(substituted amino)-1,2-dihydro-1-hydroxy-2-iminopyrimidines may also be used according to the ivention.

They may be chosen from compounds of the formula IX: 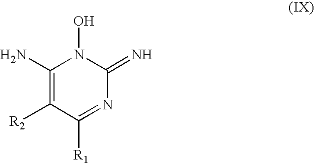
wherein R.₁ is a moiety selected from the group consisting of moieties of the formula 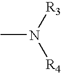
wherein R3 and R4 are selected from the group consisting of hydrogen, lower alkyl, lower alkenyl, lower aralky, and lower cycloalkyl, and taken together R3 and R4 may be a heterocyclic moiety selected from the group consisting of aziridinyl, azetidinyl, pyrrolidinyl, piperidino, hexahydroazepinyl, heptamethylenimino, octamethylenimino, morpholino, and 4-lower-alkylpiperazinyl, each of said heterocyclic moieties having attached as substituents on the carbon atoms 0-3 lower alkyl groups, hydroxy or alkoxy, and wherein R2 is selected from the group consisting of hydrogen, lower alkyl, lower alkenyl, lower alkoxyalkyl, lower cycloalkyl, lower aryl, lower aralkyl, lower alkaryl, lower alkaralkyl, lower alkoxyaralkyl, and lower haloaralkyl and the pharmacologically acceptable acid addition salts thereof.

A preffered compound is 6-amino-1,2-dihydro-1-hydroxy-2-imino-4-piperidinopyrimidine in the form of the free base or acid addition salts thereof.

6-amino-1,2-dihydro-i-hydroxy-2-iminopyrimidines, their carboxyacylated counterparts, and the corresponding acid addition salts thereof may also be used according to the invention. These substances have the formula X: 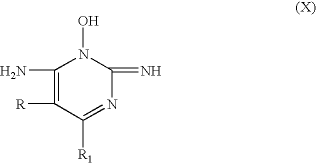
wherein R1 is a moiety selected from the group consisting of moieties of thc formula 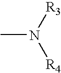
wherein R3 and R4 are selected from the group cons'isting of hydrogon, lower alkyl, lower alkenyl, lower aralkyl, and lower cycloalkyl, with the proviso that both R3 and R4 are not hydrogen, and the heterocyclic moieties, aziridinyl, azetidinyl, pyrrolidinyl, piperidino, hexahydroazepinyl, heptamethylenimino, octamethylenimino, morpholino, and 4-lower-alkylpiperazinyl, each of said heterocyclic moieties having attached as substituents on carbon atoms thereof zero to 3 lower alkyls, inclusive, a nitrogen atom of each of said heterocyclic moieties being the point of attachment of RI to the ring in said formula.

When R1 is 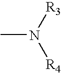
R3 and R4 can be alike or different. When R1 is a heteiocyclic moiety, the alkyls which can be attached thereto can all be different or any two or all of them can be alike.

R in Formula X is a monovalent moiety which can be the same as or dilferent than R1. Although R can be any of a large variety of atoms or groups of atoms, this invention relates especially to compounds of the formulas: 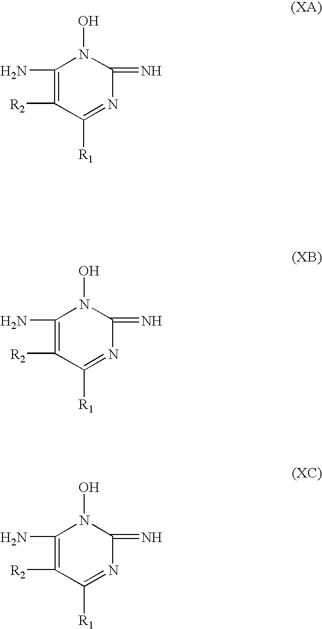
wherein, in each instance, R1 is as defined above.

In Formula XA, R is selected from the group consisting of hydrogen, lower alkyl, lower alkenyl, lower alkoxyalkyl, lower cycloalkyl, lower aryl, lower aralkyl, lower alkaryl, lower alkaralkyl, lower alkoxyaralkyl, and lower haloaralkyl. In Formula XB, R5 is selected from the group consisting of chlorine, bromine, iodine, nitroso, nitro, amino, phenylthio, lower alkylphenylthio, and halophenylthio. In Formula XC, R6 is assigned the same definition as R1, above. R6 can be the same as or different than R1, within the scope of that definition.

The novel 1,2-dihydro-1-hydroxypyrimidines of this invention can be represented by formulas other than Formulas X, XA, B and C. For example, with regard to Formula X, among such other formulas are:

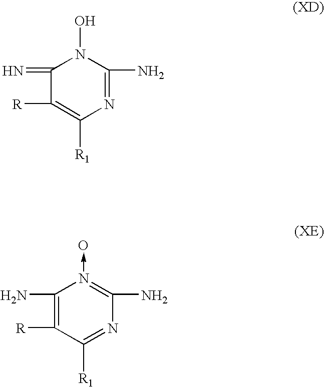

Compounds of Formulas XD and XE are tautomeric with compounds of Formula I. It is to be understood, however, that the compounds, are likely to be mixtmes of tautomeric forms, the compositions of which are dependent on such factors as the nature of R1, R2, R5, and R6, and the environment. In some instances, one form or another may predominate.

Examples of lower alkyl are methyl, ethyl, propyl, butyl, pentyl, hexyl, heptyl, octyl, and isomeric forms thereof. Examples of lower alkenyl are allyl, 1-methylallyl, 2-methylallyl (methaliyl), 2-butenyl (crotyl), 3-butenyl, 1,2-dimethylallyl, 1,1-dimethylallyl, 2-ethylallyl, 1-methyl-2-butonyl, 2-methyl-2-butenyl, 3-methyl-2-butenyl, 3-pentenyl, 2,3-dimethyl-2-butenyl, 1,1,2-trimethylallyl, 1,3-dimethyl-2-butenyl, 1-ethyl-2-butcnyl, 4-methyl-2-pentenyl, 2-ethyl-2-pentenyl, 4,4-dimethyl-2-pentenyl, 2-heptenyl, 2-octenyl, 5-octenyl, 1,4-dimethyl-4-hexenyl, and the like. Examples of lower alkoxyalkyl are 2-methoxyethyl, 2-ethoxyethyl, 2-butoxyethyl, 2-hexyloxyethyl, 2-octyloxyethyl, 2-methoxypropyl, 3-methoxypropyl, 3-propoxypropyl, 2-metltoxybutyl, 3-ethoxybntyl, 4-butoxybutyl, 2ethoxyhexyl, 3-methoxy-3-methylpentyl, 4-methoxyoctyl, and the like. Examples of lower cycloalkyl are cyclopropyl, Z-methylcyclopropyl, 2,2-dimethylcyclopropyl, 2,3-diethylcyclopropyl, 2-butylcyclopropyl, cyclobutyl, 2-methylcyclobutyl, 3-propylcyclobutyl, 2,3,4-triethylcyclobutyl, cyclopentyl, 2,2-dimethylcyclopentyl, 3-pentylcyclopentyl 3-tert-butylcyclopentyl, cyclohexyl, 4-tert-butylcyclohexyl, 3-isopropylcyolohexyl, 2,2-dimethylcyclohexyl, cycloheptyl, cyclooctyl, and the like. Examples of lower aryi are phenyl, 1-naphthyl, and 2-naphthyl. Examples of lower alkaryl are o-tolyl, m-tolyl, p-tolyl, m-ethylphenyl, p-tert-butylphenyl, the isomeric forms of xylyl, the isomeric forms of trimethylphenyl, 4-methyl-1-naphthyl, 6-propyl-2-naphthyl, 2,4,5,7-tetramethyl-1-naphthyl, and the like. Examples of lower aralkyl are benzyl, phenethyl, 1-phenylethyl, 2-phenylpropyl, 4-phenylbutyl, 6-phenplhexyl, 5-phenyl-2-methylpentyl, 1-naphthylmethyl, 2-(1-naphthyl)ethyl, 2-(2-naphthyl)ethyl, and the like, Examples of lower alkaralkyl are o-tolylmethyl, m-tolylmethyl, p-tolylmethyl, 4-tert-butylphenylmethyl, 2-(ptolyl)ethyl, 1-(m-tolyl)ethyl, 3-(o-ethylphenyl)propyl, 4methyl-1-naphthylmethyl, 6-tert-butyl-2-naphthylmethyl, and the like. Examples of lower alkoxyaralkyl are o-methoxybenzyl, m-methoxybenzyl, p-methoxybenzyl, 2 (m-methoxyphenyl)ethyl, 3-(p-ethoxyphenyl)propyl, 4(p-tert-butoxyphenyl)butyl, 4-methoxy-1-naphthylmethyl, and the like. Examples of lower haloaralkyl are o-chlorobenzyl, m-fluorobenzyl, p-bromobenzyl, 2-(m-iodo-phenyl)ethyl, 2,4-dichlorobenzyl, 6-bromo-1-naphthylmethyl, 4-(p-chlorophenyl)butyl, and the like. Examples of lower alkylphenylthio are o-tolylthio, m-tolylthio, p-tolylthio, the isomeric forms of xylylthio, p-ethylphenylthio, m-butylphenylthio, and the like. Examples of halophenylthio are p-chlorophenylthio, m-bromophenylthio, o-flnorophenylthio, 3,4-dichlorophcnylthio, and the like. Examples of heterocyclic moieties within the scopc of R1, in addition to those already mentioned above, are 2-methylaziridinyl, 2-ethylaziridinyl, 2-butylazirindinyl, 2,3-dimethylaziridinyl, 2,2-dimethylaziridinyl, 2-methylazetidinyl, 3-methylazetidinyl, 2-octylazetidinyl, 2,2-dimethyl-azetidinyl, 3,3diethylazetidinyl, 2,4,4-trimethylazetidinyl, 2,3,4-trimethylazetidinyl, 2-methylpyrrolidinyl, 3-butylpyrrolidinyl, 2-isohexylpyrmlidinyl, 2,3-dimethylpyrrolidinyl, 2,2-dimethylpyrrolidinyl, 2,5-diethylpyrrolidinyl, 3-tert-butylpyrrolidinyl, 2,3,5-trimethylpyrrolidinyl, 3,4-dioctylpyrrolidinyl, 2-methylpiperidino, 3-methylpiperidino, 4-methylpiperidino, 3-isopropylpiperidino, 4.tert-butylpiperidino, 2-methyl-5-ethylpiperidino, 3,5-dipentylpiperidino, 2,4,6-trimethyipiperidino, 2,6-dimethyl-4-octytpiperidino, 2,3,5-triethylpiperidino, 2-ethylhexahydroazepinyl, 4-tertbutylhexahydroazcpinyl, 9-heptylhexahydroazepiuyl, 2,4-dimethylhexahydroazepinyl, 3,3-dimethylhexahydroazepinyl, 2,4,6-tripropylhexahydroazepinyl, 2-methylheptamethylenimino, 5-butylheptamethylenimino, 2,4-diisopropylheptamethylenimino, 3,3-diethylheptamethylenimino, 2,5,8-trimethylheptamethylenimino, 3-methyloctamethylenimino, 2,9-diethyloctamethylenimino, 4-isooctyloctamethylenimino, 2-ethylmorpholino, 2-methyl-5-ethylmorpholino, 3,3-dimethylmorpholino, 2,6-ditert butylmospholino, 4-methylpiperazinyl, 4-isopropylpiperazinyl, and the like. In each of the above examples of heterocyclic moieties, the free valence, and hence the point of attachment to a carbon atom of the pyrimidine ring, is at the heterocyclic nitrogen atom.

Preferably the active substance is chosen from 6-amino-1,2-dihydro-1-hydroxy-2-iminopyrimidines, their carboxyacylated counterparts, and the corresponding acid addition salts thereof, preferably the one that are substituted in the 4-position and optionally in the 5-position, wherein the substituent in the 4-position being a secondary or tertiary amino moiety analogues and salts thereof. Minoxidil, 6-amino-1,2-dihydro-1-hydroxy-2-imino-piperidinopyrimidine, and it's analogues are preferred.

All compounds of forms (I-X), may be transformed into their cosmtecially and pharmaceutically acceptable acid additions salts, such as acides sulfuric acid, chlorhydric acid, bromhydric acid, phosphoric acid, acetic acid, benzoic acid, salicylice acid, glycolic acid, succinic acid, nicotinic acid, tartric acid, maleic acid, pamoic acid, methan sulfonie acid, picric acid, lactic acid, etc . . .

17β-N-(monosubstituted)-carbamoyl-4-aza-5.alpha.-androsten-1-en-3 one compounds may also be used according to the invention.

These compounds have the formula XI: 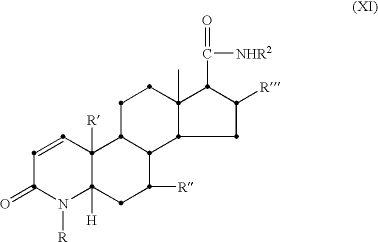
wherein R is hydrogen, methyl or ethyl.

R2 is a hydrocarbon radical selected from straight and branched chain alkyl of from 1-12 carbons or monocyclic aryl optionally containing 1 or more lower alkyl substituents of from 1-2 carbon atoms and/or more halogen (Cl, F or Br) substituents.

R′ is hydrogen or methyl.

R″ is hydrogen or β-methyl.

R′″ is hydrogen, α-methyl or β-methyl.

A preferred embodiment of the compounds is represented by the formula: 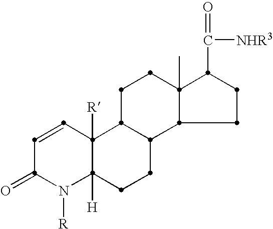
wherein R is hydrogen, methyl or ethyl, and R.sup.3 is branched chain alkyl of from 4-8 carbons.

Representative compounds of the present invention include the following:
17β-N-tertbutylcarbamoyl)-4-aza-4-methyl-5.alpha.-androst-1-en-3-one,
17β-(N-isobutylcarbamoyl)-4-aza-4-methyl-5.alpha.-androst-1-en-3-one,
17β-N-tert-octylcarbamoyl)-4-aza-4-methyl-5.alpha.-androst-1-en-3-one
17β-N-octylcarbamoyl)-4-aza-4-methyl-5.alpha.-androst-1-en-3-one,
17β-.-(n-1,1-diethylbutylcarbamoyl)-4-aza-4-methyl-5-.alpha.-androst-1-en-3-one,
17β-.-(N-neopentylcarbamoyl)-4-aza-4-methyl-5.alpha.-androst-1-en-3-one,
17β-N-tert-amylcarbamoyl-4-aza-4-methyl-5.alpha.-androst-1-en-3-one,
17β-(N-tert-hexylcarbamoyl)-4-aza-4-methyl-5.alpha.-androst-1-en-3-one
and the corresponding compounds wherein the 4-methyl substituent is replaced in each of the above named compounds by a hydrogen or an ethyl radical.

Also included as representative compounds are any of the above indicated compounds having the N-branched chain substituent replaced by a methyl ethyl, propyl, i-propyl, butyl, phenyl; 2, 3 or 4 toxyl, xylyl, 2-bromo or 2-chlorophenyl, 2-6-dichloro, or a 2,6-dibromophenyl substituent.

Specially preferred is Finasteride, marketed under the tradename of PROSCAR™, by Merck & Co., Inc., which is 17β-(N-tert-butyl carbamoyl)-4-aza-5 alpha-androst-1-en-3-one, see U.S. Pat. No. 4,760,071 (1988).

In one preferred embodiment of the preparation, the lipophilic components are chosen among fatty acids with fourteen to twenty carbon atoms or any mixtures thereof and dimethylpolysiloxane (dimethicone), the hydrophilic components being chosen among triethanolamine, monopropylene glycol, glycerol, sorbitol, polyethylene glycol and polyvinyl pyrrolidone.

The preparation may be prepared from one lipophilic component comprising said fatty acid or a mixture thereof, and one hydrophilic component comprising triethanolamine, the molar ratio of fatty acid (mixture): triethanolamine preferably being higher than 1.

In another preferred embodiment, the preparation is prepared from one lipophilic component, which is a mixture of fatty acids of the following composition: at most about 2% of a component consisting of a chain of fourteen carbon atoms, between about 47 and about 52% of a component consisting of a chain of 16 carbon atoms, between about 43 and about 48% of a component consisting of a chain of 18 carbon atoms and at most about 1% of a component consisting of a chain of twenty carbon atoms. All percentages given in the present application are indicated in weight/weight.

In one alternative embodiment, said fatty acid consisting of a chain of fourteen to twenty carbon atoms, or said mixture of such fatty acids, may be of any other composition, for example containing an essential portion of myristic acid, such as about 90% myristic acid or an essential portion of palmitic acid, such as about 90% palmitic acid. Thus, in the present context “a fatty acid consisting of a chain of fourteen to twenty carbon atoms, or a mixture of such fatty acids”, refers to any suitable mixture of higher fatty acids. However, a predominant portion of a fatty acid consisting of a chain of 18 carbon atoms is most preferred.

Thus, the most preferred embodiment of the preparation is prepared from a ratio between a fatty acid mixture, having the above defined preferred composition, and triethanolamine higher than about 2:1, preferably higher than about 3:1, such as about 3:6:1. Other advantageous embodiments of the skin preparation according to the present invention are prepared from the same ratio between any equivalent lipophilic component and triethanolamine.

It has been shown to be advantageous to adapt the ratio between the primary lipophilic components, i.e. the fatty acid consisting of a chain of 14 to 20 carbon atoms or the mixture of such fatty acids, preferably of the above defined composition, and triethanolamine to enable a certain portion of the acid in the final skin preparation to form a salt with the triethanolamine, while another portion exists as free fatty acid. The presence of both the free fatty acid and the salt in the final product, resulting from the above defined preferred ratios between the mixture of fatty acids and the triethanolamine, may be one reason to the unique properties of the skin preparation according to the invention, i.e. that it acts both in the depth of the skin and creates the osmotic membrane, which is new per se, and, additionally, that it simultaneously is capable of protecting the surface of the skin against influences of deleterious substances. These specific properties of the skin preparation according to the invention are also presumably due to the particular method of preparation thereof, which will be described in more detail below.

In one particular embodiment, the preparation is prepared from a fatty acid consisting of a chain of 14 to 20 carbon atoms, or any mixture of such fatty acids, triethanolamine, monopropylene glycol, polyvinyl pyrrolidone, dimethyl polysiloxane and a component which is chosen among glycerol, sorbitol, and polyethylenglycol, or a mixture thereof, as well as water.

As concerns the choice of glycerol, sorbitol or poylvinyl pyrrolidone, the primary purpose of this component is to enable the skin preparation according to the invention to be quickly absorbed by the skin. In general, the component will reside in the more humid parts of the horny layer of the epidermis. In a simple embodiment thereof, the skin preparation according to the invention is prepared from glycerol, whereas in alternative embodiments it is prepared from sorbitol or polyethylene glycol, such as PEG 200, in equivalent amounts. In other embodiments, the preparation is prepared from a mixture of MPG sorbitol and PEG or any other suitable mixture of the above-defined components.

As regards the ingredients used in the manufacture of the preparation some suitable examples thereof are as follows. The triethanolamine may e.g. be 99LFG85 (MB-Sveda), the fatty acid component may e.g. be Safacid 16/18CR^{R} (Vendico Chem) and the polyvinyl pyrrolidone may e.g. be PVP K30 (ISP).

One preferred embodiment of the preparation is prepared from about 5 to 8% of a fatty acid consisting of a chain of 14 to 20 carbon atoms, or a mixture of such fatty acids, e.g. the preferred mixture defined above, about 0.73-2.66% triethanolamine, about 4.5-7.0% monopropylene glycol, about 1-2.5%, preferably 1.5-1.97% polyvinyl pyrrolidone, at most about 5%, such as about 1-2.5% and preferably about 0.5-0.95%, dimethyl polysiloxane and about 1.5-2% glycerol, sorbitol or polyethylene glycol, or any mixture thereof, the balance being water up to 100%. According to an especially advantageous embodiment, the skin preparation according to the invention is prepared from about 6.25% of a fatty acid consisting of a chain of 14 to 20 carbon atoms, or any suitable mixture of such fatty acids, e.g. the preferred mixture as defined above, about 0.91% triethanolamine, about 5.8% of monopropylene glycol, about 1.96% polyvinyl pyrrolidone, about 0.89% dimethyl polysiloxane and about 1.52% of glycerol, sorbitol or polyethylene glycol, or any mixture thereof, the balance being water. As regards the portion of dimethyl polysiloxane, it is noted that even though the preferred percentages are as indicated above, it may be any value within the region of from 0% and up to about 5%, depending on the other ingredients.

The preparation may be prepared from polyvinyl pyrrolidone (PVP), a preferred average molecular weight being about 8,000-63,000 Dalton, preferably about 38,000 Dalton. The dimethyl polysiloxane (dimethicone), from which the preparation is prepared, exhibits an exemplary viscosity of from about 100-1,000 cp, preferably about 350 cp. Anyway, the polymers from which the preparation is prepared will exhibit a high enough level of polymerisation not to penetrate the horny layer of the epidermis of the skin.

In one embodiment, the preparation includes a frothing agent, e.g. Polysorbate 20. If present, said frothing agent is used in an amount of about 1.3-2.7%, preferably about 1.5%.

In another embodiment, the preparation comprises a preservative and/or an aromatic agent, the preservative being present in an amount of about 0.5% and the amount of the aromatic agent being about 0.05%-0.5%, preferably about 0.18%. However, said amount is dependent upon the kind of aromatic agent chosen. One preferred preservative according to the invention is Phenonip, which is well known within this field.

The composition according to the invention may be for cosmetical or pharmaceutical use. Thus, the invention relates to a composition for use as a medicine. It also relates to the use of the composition for the preparation of a pharmaceutical against hair loss of different origin.

Therefore, the amount of active compound may be high such as e.g. from 0.05 to 10% by weight, or high such as from 10 to 50% by weight.

The percentage by weight of the active compound in the composition typically ranges from about 0.1% to about 20.0% of the preparation, preferably from about 1% to about 10%, e.g. from 2% to 7%, 0.1-6% and especially 5% by weight.

The compositions encompassed by this invention include formulations adapted for topical application to the human scalp. Conventional pharmaceutical preparations for this purpose include ointments, lotions, pastes, jellies, gels, mousses, sprays, foams, aerosols, and the like. The term “ointment” embraces formulations, which include creams, which are either oil-in-water or water-in-oil emulsions. The compounds may also be formulated into liposomal preparations or lipid emulsions or dissolved in conventional solvents such as alcohol, propanol, and the like.

The pharmaceutical preparations of the subject invention are applied on a regular basis, with or without occlusion, for a period of time sufficient to effect hair growth. Occlusion of the preparation may be obtained by any conventional means such as bandages, plastic coverings, shower caps swimming caps, etc. The percentage of active ingredients as well as frequency of application may be varied as necessary or desirable to achieve the desired results.

The formulation should be substantive to the scalp, i.e., will not run off the scalp. Therefore, the preferred compositions are in the form of gels, ointments or creams, where the active ingredients may remain at the site of application for extended period of time.

Suitable additives that may be admixed with the active substance such as e.g. minoxidil include, but are not limited to solvents, such as water, glycols, esters, alcohols, lipid materials, coloring agents, fragrances, anti-oxidants, thickening agents, ultra-violet light stabilizers, and other additives accepted in pharmaceutical formulations.

In the most preferred embodiment of the preparation, the two phase preparation according to the invention is prepared to enable application thereof as a foam, for example, from an aerosolic container. The choice of propellant added to create such a product will be dependent on the intended use and may easily be done by a person skilled within this area.

Different propellants such as propane or butane are used depending on the intended use of the preparation. However, at present there are indications that butane, or a mixture wherein butane is predominant, is the most advantageous propellant, even though other equivalent alternatives may be used if deemed suitable for a particular aplication. A person skilled within this field may well make an appropriate choice of a suitable propellant for each situation and application.

A preparation that enables the formation of an osmotic or semi-permeable membrane in the skin, may be produced by the following steps:
a) the dissolution in water of a fatty acid consisting of a chain of 14 to 20 carbon atoms, or a mixture of such fatty acids, in a vessel during careful agitation to provide Solution A;
b) the dissolution, in another vessel, of polyvinyl pyrrolidone in water and during powerful agitation;
c) the addition, during agitation, of monopropylene glycol, triethanolamine and glycerol, sorbitol or polyethylene glycol, or any mixture of those three last mentioned; to the product obtained from step b), whereby Solution B is obtained after a reaction time t_{c};
d) the feeding of Solution B to the vessel containing Solution A, whereafter the contents thereof are allowed to react during a reaction time t_{d}; and,
e) the cooling of the product from step d) by the addition of cold water during continued agitation, the dimethylpolysiloxane being added and the total amount being balanced with water.

According to one particular embodiment of the process as defined above, a frothing agent, an aromatic agent and/or a preservative are also added, preferably together with the dimethyl polysiloxane. These further additives may be of different kinds, proportions and amounts, such as the ones already disclosed above in connection with the skin preparation according to the invention.

Step a) and/or step b) of the process are preferably performed at a higher temperature than about 80° C., such as about 95° C. According to an advantageous embodiment, the above defined times t_{c} and t_{d} are about 30 minutes and 60 minutes, respectively.

The process may be performed in any conventional vessel, while step b), however, preferably is performed in a high-speed double-mounted turbine dissolver. The agitation is provided e.g. by a propeller. As concerns other measures, equipments, reagents etc. in connection with the process according to the invention, a person skilled within this area will easily make the requisite choices and judgements thereof to achieve the desired results.

According to one embodiment of the process, a mixture of fatty acids consisting of chains of 14 to 20 carbon atoms is used, which exhibits the following composition: at most about 2% of a component consisting of a chain of 14 carbon atoms, between about 47 and about 52% of a component consisting of a chain of 16 carbon atoms, between about 43 and about 48% of a component consisting of a chain of 18 carbon atoms and at most about 1% of a component consisting of a chain of 20 carbon atoms. Alternative compositions of said mixture of fatty acids consisting of chains of 14 to 20 carbon atoms are already mentioned above in connection with the skin preparation according to the invention.

In an advantageous embodiment, the process according to the invention relates to the manufacture of a skin preparation, which comprises about 5 to 8%, preferably about 6.25%, of a fatty acid of 14 to 20 carbon atoms, or a mixture of such fatty acids, about 0.73% to 2.66%, preferably about 0.91% of triethanolamine, about 4.5 to 7.0%, preferably about 5.8%, of monopropylene glycol, about 1-2.5%, such as about 1.5 to 1.97%, preferably about 1.69%, of polyvinyl pyrrolidone, anywhere between 0 and 5%, such as about 0.5 to 0.95%, preferably about 0.89%, of dimethyl polysiloxane and about 1 to 2%, preferably about 1.52%, of a component, which may be anyone of glycerol, sorbitol and polyethylene glycol, or any mixture thereof, the rest being balanced with water up to 100%. Specifically preferred ratios between the lipophilic and hydrophilic components are as already discussed above in connection with the preparation.

The invention also relates to a process for the manufacture of the composition, whereby the preparation is prepared by dissolving the lipophilic components in water in a separate container to be combined with hydrophilic components, which have been blended and brought to react in another vessel, the active substance is dissolved in a solvent and mixed with the preparation. The active substance may be dissolved in the hydrophilic phase and/or in the lipophilic phase.

Thus, the active substance may be dissolved in one of the hydrophilic components of the preparation such as triethanolamine, monopropylene glycol, glycerol, sorbitol, polyethylene glycol and polyvinyl pyrrolidone.

It may also be mixed with or dissolved in the lipophilic component such as fatty acids with fourteen to twenty carbon atoms or any mixtures thereof or dimethylpolysiloxane (dimethicone).

The invention also relates to a method of treating humans for hair loss, which comprises administration to a human susceptible to hair loss of an effective amount of a composition according to the invention. The composition is preferably topically applied to the scalp. The composition may be applied one or several times per day, preferably once a day.

In an effort to improve the efficacy of topical minoxidil compositions for the treatment of hair loss, a more effective method for applying minoxidil and transporting it across the skin barrier has been developed. The present method requires fewer applications of minoxidil per day i.e., once per day treatment, and provides for less systemic side effects. Further the composition has good cosmetical properties and there are no skinirritations observed. The product according to the invention may be used with lower doses than menoxidil and still the hair growth is the same.

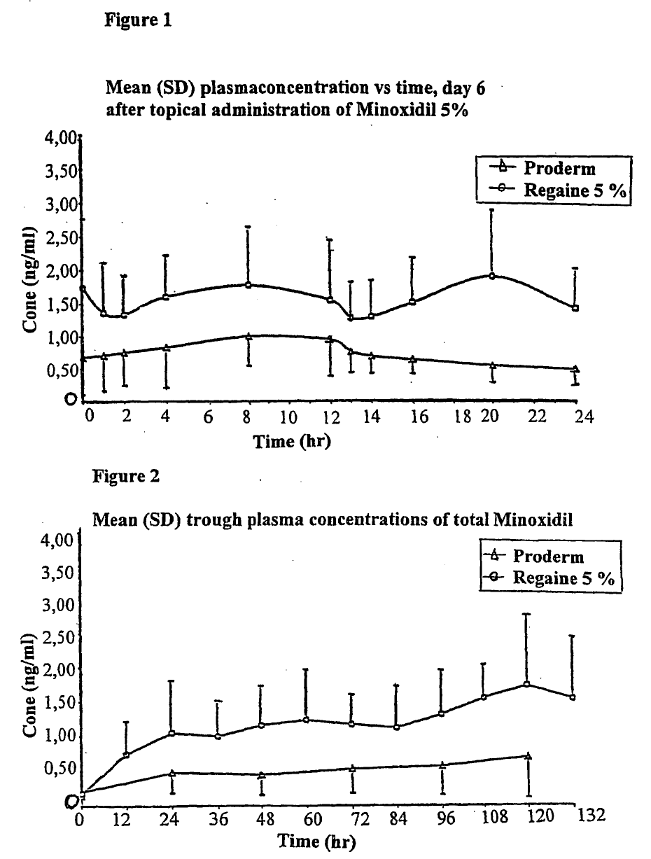

The systemic uptake of minoxidil after once daily application of three different formulations according to the invention (Proderm) has been compared to the systemic uptake after twice daily application of Regaine® 5%. The tested once-daily treatment Proderm had a significantly lower maximum observed plasma concentration compared to the twice-daily treatment (Regaine® 5%)(Example 7, FIG. 1 and 2).

The tested once daily treatment Proderm had significantly lower rate and extent of absorption compared to the twice-daily treatment (Regaine® 5%). No differences between the treatments were noticed regarding local tolerance or hair growth.

### DETAILED DESCRIPTION OF THE DRAWINGS

FIG. 1 shows mean (SD) plasma concentrations vs time, day 6 after topical administration of Minoxidil 5% in a composition according to the invention Proderm-Δ-and Regain-O—.

FIG. 2 shows mean (SD) through plasma concentrations of total Minoxidil. FIG. 1-2 show mean (SD) steady-state and trough plasma concentration versus time curves after topical administration of Minoxidil for six days.

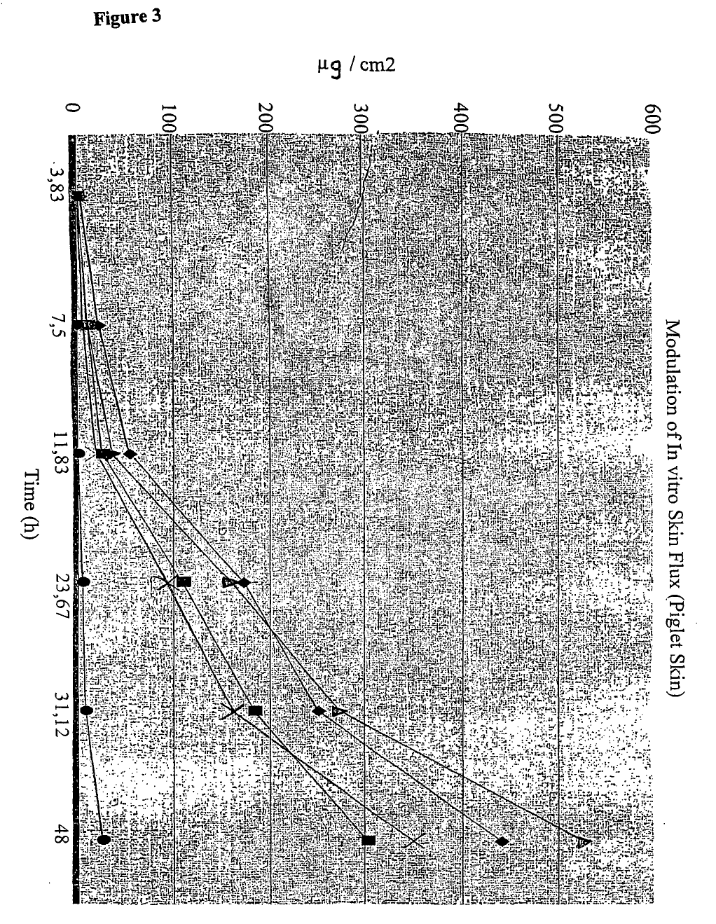

FIG. 3 shows the permeation of minoxidil from a composition according to the invention Proderm (▴, ▪, X) compared to Rogaine (●).

Throughout this specification and the claims, the words “comprises” and “comprising” and “comprised” are used in a non-exclusive sense. All cited references are incorporated by reference.

### EXAMPLES

Below, examples of the composition and preparation according to the invention are disclosed by the ingredients and manufacture thereof. Also, the advantageous results of several tests performed at institutions acknowledged within this area are reported. In the experimental descriptions that follows, references are sometimes made to complete reports produced by the institution in question, which reports are hereby incorporated herein by reference. It is stressed that these examples are only construed to illustrate the invention and that they are not to be interpreted as limiting the scope of protection as defined by the claims in any way.

### Example 1

### Preparation

A preparation was prepared from the following ingredients in the amounts indicated below:

| Ingredient: | Amount, % (w/w) |
|---|---|
| Stearic acid | 6.56 |
| Monopropylene glycol (MPG) | 6.09 |
| Polyvinyl pyrollidone (PVP) | 2.0 |
| Glycerol | 1.78 |
| Polysorbate 20 | 1.78 |
| Triethanolamine (TEA) | 1.1 |
| Dimethicone | 0.95 |
| Preservative | 0.5 |
| Water | to 100 |

Process of Preparation

Phase 1 is prepared by warming about ⅓ of the water to about 95° C. in a vessel equipped with propeller agitation. The steraric acid is added to the water during agitation.

Phase 2 is prepared by dissolving PVP in water at a temperature of about 95° C., in a double mounted turbine dissolver during energetic agitation. Then, MPG, glycerol and TEA are added and the mixture is blended during about 30 minutes.

Phase 2 is then supplied to Phase 1 and the two phases are agitated during 1 h. The mixture is cooled by the addition of cold water during continued agitation. Finally, Polysorbate 20 and an eventual preservative (Phenonip) are added, The total amount is balanced up to 100% by the addition of water.

The resulting liquid product including the two phases is blended with a requisite amount of a suitable propellant and it is supplied to a suitable aerosolic container, from which the skin preparation may be withdrawn as foam. Its non-sticky character and the foam form makes it easy to distribute over the desired area of skin and pleasant to wear.

### Example 2

### Preparation

Below, the best mode of the preparation is disclosed, as is known by the inventor at the filing of the present application. Thus, a skin preparation was prepared from the following ingredients in the amounts indicated below:

| Ingredient: | Amount, % (w/w) |
|---|---|
| Stearic acid | 6.25 |
| Monopropylene glycol (MPG) | 5.8 |
| Polyvinyl pyrrolidone (PVP) | 1.9 |
| Glycerol | 1.7 |
| Polysorbate 20 | 1.4 |
| Triethanolamine (TEA) | 1.05 |
| Dimethicone | 0.95 |
| Preservative | 0.5 |
| Water | to 100 |

Process of Preparation

The present skin preparation was prepared as disclosed above in example 1. A soft and pleasant foam was obtained, which is excellent for use to treat skin irritations and rashes. It is also suitable to use as a carrier of an active substance, e.g. UV absorbants in order to create a sun screen preparation. Examples of other active substances may be any substance that has a pharmaceutical effect and, thus, the present preparation may be used for a topic administration of medicaments.

In addition, the present skin preparation also made the skin on which it was applicated resistent to concentrated sulphuric acid. Thus, said acid could be contacted with the hands treated with the skin preparation without causing any disagreeable feeling or lesion.

### Example 3

### Preparation

A preparation was prepared from the following ingredients in the amounts indicated below:

| Ingredient: | Amount, %(w/w) |
|---|---|
| Stearic acid | 7.8 |
| Monopropylene glycol (MPG) | 6.8 |
| Polyvinyl pyrrolidone (PVP) | 1.5 |
| Glycerol | 1.0 |
| Polysorbate 20 | 1.34 |
| Triethanolamine (TEA) | 1.9 |
| Dimethicone | 0.5 |
| Preservative | 0.5 |
| Water | to 100 |

Process of Preparation

The preparation was prepared as disclosed above in example 1. A soft foam was obtained, which was easily absorbed by the skin and pleasant to wear. The skin felt flexible after application thereof, especially after a repeated use.

### Example 4

### Composition of Preparation Containing Minoxidil

4.5 g of Minoxidil was dissolved in 25.5 g propylen glycol heated to 80° C. Proderm skin protector, produced according to example 3 containing 5.8 weight % propylene glycol, was heated to 80° C. The solution containing Minoxidil was added under stirring to 60 g of the Proderm skin protector. The mixture was allowed to cool to 25° C. and filled into cans in portions of 90 g. The content of Minoxidil was 5 weight % and of propylene glycol 28 weight %.

### Example 5

### Composition Preparation Containing Minoxidil

1.5 g of Minoxidil was dissolved in 8.5 g propylen glycol heated to 80° C. Proderm skin protector, produced according to example 3 containing 5.8 weight % propylene glycol, was heated to 80° C. The solution containing Minoxidil was added under stirring to 20 g of the Proderm skin protector. The mixture was allowed to cool to 25° C. and filled in portions of 30 g into aerosol packages containing 3 g propan/butan as propellant gas. The content of Minoxidil was 5 weight % and of propylene glycol 28 weight %.

### Example 6

### Composition of Preparation Containing Minoxidil

5 g of Minoxidil was dissolved in 26 g propylen glycol heated to 80° C. Proderm skin protector, produced according to example 3 containing 5.8 weight % propylene glycol, was heated to 80° C. The solution containing Minoxidil was added under stirring to 60 g of the Proderm skin protector. The mixture was allowed to cool to 25° C. and filled into cans in portions of 91 g. The content of Minoxidil was 5.5 weight % and of propylene glycol 28 weight %.

### Example 7

### Effect of a Composition According to the Invention Compared with Minoxidil in the Form of Regain

The rate and extent of systemic minoxidil absorption following once-daily topical dosing of a novel 5% minoxidil solution and twice-daily topical dosing of Regaine® 5% solution was compared. The local tolerance of the different treatments was also studied.

The following abbreviations and definitions are used:

| | |
|---|---|
| AE | Adverse Events |
| AUC₀₋ₜ | Area under the plasma concentration-time curve from zero to last |
| | sampling time |
| BMI | Body Mass Index |
| Cₘₐₓ | Maximum observed plasma concentration |
| CRF | Case Report Form |
| GCP | Good Clinical Practice |
| IEC | Independent Ethics Committee |
| HPLC | High Performance Liquid Chromatography |
| MNX | Minoxidil |
| MPA | Medical Products Agency |
| NEC | Not Elsewhere Classified |
| NOS | Not Otherwise Specified |
| SAE | Serious Adverse Events |
| Tₘₐₓ | Time of the observed maximum plasma concentration |
| UV | Ultra Violet |

Overall Study Design and Plan

The present investigation was an open, multiple dose, randomised, four-way crossover study. The subjects were healthy male volunteers, 18-55 years old, with type III vertex, type IV or type V pattern baldness according to the Hamilton Scale. The test treatments were three novel 5% minoxidil topical solutions for once-daily administration and Regaine® 5% topical solution as a reference product, administered twice-daily. All subjects received all four treatments with at least one week of washout between the treatments. One ml of the solution was applied to 100 cm² of bald scalp by the personnel once or twice daily during six consecutive days. Blood samples were drawn for determination of total minoxidil concentration in plasma immediately before each dose (through values) and at ten time points during 24 hours after the morning dose of day 6. Occurrence of any local irritation, itching, redness, dryness or folliculitis was recorded before and 1 hour after the first and last morning dose. Pulse and blood pressure measurements were performed before the morning application of day 1 and day 6.

A cross-over design was chosen. This design has the following advantages when comparing treatments; Each subject serves as his own control; It allows a within subject comparison between formulations; It removes the inter-subject variability from the comparison between formulations; With a proper randomisation of subjects to the sequence of formulation administration, it provides the best unbiased estimates for the differences (or ratios) between treatments.

The study was open, as the primary objective was to study the systemic exposure of minoxidil following the different treatments.

Study Population

The subjects included were healthy people without medication. Other therapy considered necessary for the subject's welfare was allowed at the discretion of the Investigator.

Inclusion criteria were: Male, 18 to 55 years of age; Healthy according to the health examination and medical history; BMI<30; Type III vertex, type IV or type V pattern baldness according to the Hamilton Scale; Willing to provide informed consent.

Exclusion criteria were: History of significant allergy; Any current or past medical condition or use of drugs, which might significantly affect the response to the administered drug; Participation in another clinical trial within 1 month prior to the start of the trial; Blood donation within 3 months prior to the start of the trial.

A subject was to be withdrawn from the trial treatment if, in the opinion of the investigator, it was medically necessary, in the event of non-compliance with the protocol, or if it was the wish of the subject.

The subjects were given instructions not to: Wash his hair at any other time than scheduled (prior to each application); Use hair oil, sun lotions, hair mousse or similar products for 24 hours prior to throughout the study; Expose the head to sun (including solarium) during the application time; Use hairdryers; Wear hairpieces; Engage in any strenuous activities; Go swimming or take a sauna; Eat hot or spicy food.

An open study design was chosen for practical reasons.

Products used for Treatments

Unknown=Treatment A

Unknown=Treatment B

Minoxidil 5%/Proderm (preparation according to example 4)=Treatment C

Regaine® 5% topical solution=Treatment D (Reference product)

The study products were three novel topical solutions with minoxidil 5% and Regaine® 5% topical solution as a reference product. Minoxidil/Proderm and Regaine® 5%. The study products were manufactured at Pharmaceutical R&D, Pharmacia AB, Consumer Healthcare, Helsingborg, Sweden.

Method of Assigning Subjects to a Treatment Group

The treatments were given according to a repeated 4×4 standard Latin square, Table 1.

TABLE 1

Period

| Sequence | 1 | 2 | 3 | 4 |
|---|---|---|---|---|
| 1 | A | B | C | D |
| 2 | B | D | A | C |
| 3 | C | A | D | B |
| 4 | D | C | B | A |

A randomisation list with sequence 1-4 was created using ADLS (Almedica Drug Label System) version 5.3, at Pharmaceutical R&D, Pharmacia AB, Consumer Healthcare, Helsingborg, Sweden. The subjects included were given a number between 1 and 16 from the randomisation list.

Doses, Timing and Sample Size

The recommended dose for the reference product Regaine® 5% is 1 ml solution (=50mg). The same dose was chosen for the test treatments. All subjects received the same dose.

The prescribed dosing for Regaine® 5% is application of one ml morning and evening. The test solutions were applied only once daily since this was the aim of the project. Doses were applied at 8 am (test solutions) or at 8 am and 8 pm (reference solution) for six consecutive days to achieve steady-state plasma levels.

No formal calculation of the sample size has been performed. The sample size was chosen to 16 subjects for practical reasons. Results from previous studies indicate a SD of about 3 ng/ml for the variable Cₘₐₓ. With this assumption 16 subjects are sufficient to estimate a mean difference of 2.98 ng/ml between two treatments.

Efficacy, Safety Variables and Pharmacokinetic Assessment

Blood Sampling

Venous blood samples (5 ml) were collected in heparinized glass tubes from an antecubital vein before each application and at the following time points after the morning dose of day six: 1, 2, 4, 8, 12, 13, 14, 16, 20, and 24 hours. The blood samples were immediately chilled in ice water and centrifuged (within 60 minutes) at 1000 g at +4° C. for 10 minutes. Plasma was transferred to cryo tubes, which were stored in a −20° C. freezer pending analysis. During transportation of samples to Analytical R&D, Pharmacia AB, Consumer Healthcare, Helsingborg, Sweden, samples were kept on ice in an insulated box.

Drug Concentration Measurements

The plasma samples were stored and analysed at the Bioanalytical laboratory, Pharmacia AB, Consumer Healthcare, Helsingborg, Sweden. Determination of total minoxidil was performed by HPLC with UV detection at 280 nm, after enzymatic hydrolysis of minoxidil glucuronide using β-glucuronidase. (Internal Method No: NM-2037). The calibrated range for plasma concentrations was 0.37-20.92 ng/ml. The limit of quantification (LOQ) for the method was 0.35 ng/ml. The inter-assay bias was 8.9% and inter-assay precision (CV%) was 24.4%.

Pharmacokinetic Calculations

The pharmacokinetic calculations were performed using the software WinNonlin Professional, version 3.1 (Pharsight Corporation, CA, USA), non-compartmental analysis. Individual data was used in the calculations. Deviations from sampling times of more than 1 minute were corrected for in the pharmacokinetic calculations.

As this study included different dosing regimens (once-daily or twice-daily) a 24 hour interval at steady-state was used in making comparisons between the different treatments. The maximum observed concentration, Cₘₐₓ and the corresponding time, Tₘₐₓ after each administration on day 6 was obtained from the individual plasma concentration versus time curves. The larger of the Cₘₐₓ values following twice-daily dosing was used when comparing Cₘₐₓ values between the once-daily and twice-daily treatments. The area under the plasma concentration-time curve from time 0 to the last sampling time, AUC₀₋ₜ, was calculated by the linear trapezoidal method. The AUC₀₋₁₂ₕ values calculated at steady-state following morning and evening dosing in the twice-daily treatment group were pooled in calculating AUCₒ₋₂₄ₕ. The AUCₒ₋₂₄ₕ values were used when comparing the different once-daily test treatments to the twice-daily reference treatment D.

Concentration values below the limit of quantification were set to LOQ/2. Concentrations denoted “<value”, (too small sample volume) were excluded from the calculations.

Local Tolerance

The degree of experienced local irritation and itching was recorded using a 4-grade scale (0=none, 1=mild, 2=moderate, 3=severe) before and 1 hour after the first morning application and before and 1 hour after the last morning application (day 6).

Occurrence (yes/no) of redness, dryness, and folliculitis was evaluated by the study personnel before and 1 hour after the first morning application and before and 1 hour after the last morning application.

Safety Variables

Data on adverse events were collected by means of spontaneous reporting or observation of such events.

Pulse and Blood Pressure

Pulse and systolic and diastolic blood pressure (in the sitting position after at least 5 minutes of rest) were recorded before morning application of day 1 and 6. An automatic wrist watch meter was used.

Data Quality Assurance and Statistical and Analytical Methods

Audits of selected clinical investigator sites were conducted to assess and help assure compliance with Good Clinical Practice and applicable regulatory requirements. Statistical Methods Planned in the Protocol and Determination of Sample Size.

For all trial variables descriptive statistics were tabulated, showing mean, standard deviation, median, min and max for each treatment. Also for the primary variables, Cₘₐₓ and AUC₀₋ₜ, descriptive statistics were tabulated for the difference between the treatments. For Tₘₐₓ the frequency distribution was tabulated for each treatment.

Differences in Cₘₐₓ and AUC₀₋ₜ between treatments were tested with Wilcoxon's signed rank test. No formal adjustments for multiplicity were made, but p-values were presented for each test allowing for a relevant interpretation.

For the degree of experienced local irritation and itching and occurrence (yes/no) of redness, dryness and folliculitis the frequency distribution was tabulated for each treatment.

Results

Disposition of Subjects and Protocol Deviations

Sixteen subjects were randomised for the four different treatments and fourteen subjects completed all four treatments. Subject No.2 did not receive treatment C and subject No.16 did not receive treatment D.

There were no protocol deviations related to the study inclusion or exclusion criteria. The use of a specific shampoo, Pregaine, for washing the scalp area before each treatment were substituted to the subjects own shampoo containing no conditioner or silicon, due to difficulties in obtaining this shampoo. The application area was marked by a plastic frame with the size of 10×10 cm² instead of a surgical marker. One subject forgot to shampoo his scalp before the morning application on study day 3 (treatment D). This is not considered to have affected the study results.

There were no deviations from the protocol in the assessment of the subjects.

For treatment C subject No.6 was excluded from pharmacokinetic evaluation due to too few measurable minoxidil concentrations. Subject No.2 did not receive treatment C and subject No.16 not treatment D. All other data sets from the included subjects were used in the evaluation of the study.

16 male volunteers were included in the study. The subjects had a mean age of 37 years (range 23-54 years), weighed 80.0 kg (range 67.7-95.6 kg) and had a mean length of 178 cm (range 168-191 cm). The mean BMI was 25.3 (range 21.0-29.5 kg/m^{2).}

The bottle containing the test solution was weighed before and after each finished treatment session.

Plasma Pharmacokinetics

The mean ±SD steady-state plasma concentration time profiles for the treatments following the dose on day 6 is shown in FIG. 1. The attainment of steady-state was confirmed by visual inspection of trough plasma minoxidil concentrations (samples taken prior to each dose) day 1 to 6 (FIG. 2).

The mean observed Cₘₐₓ value was 1.38±0.64 ng/ml for the Proderm formulation. For treatment with Regaine® that was dosed twice daily the mean observed Cₘₐₓ was 2.31±1.14 ng/ml. The mean AUC₀₋₂₄ₕ for the test treatment was 18.0±6.67 ng*h/ml and for the reference twice daily treatment D 37.9±16.6 ng*h/ml. The median Tₘₐₓ value for both treatments were 8.0 hours. The mean pharmacokinetic parameters for the different treatments are summarised in Table 2. There was a significant difference in Cₘₐₓ and ACU₀₋₂₄ₕ between the Proderm test treatment and the reference treatment, Regaine® 5%.

Treatment 1=Minoxidil 5%/Proderm, once daily

Treatment 2=Regaine® 5% twice daily

TABLE 2

Treatment

| | 1 | 2 |
|---|---|---|
| Cmax (ng/ml) | | |
| Mean | 1.38 | 2.31 |
| SD | 0.643 | 1.14 |
| Min | 0.41 | 0.70 |
| Median | 1.21 | 2.25 |
| Max | 2.49 | 4.11 |
| AUCO-24 h (hr * ng/ml) | | |
| Mean | 18.02 | 37.91 |
| SD | 6.67 | 16.62 |
| Min | 5.08 | 13.78 |
| Median | 18.14 | 36.41 |
| Max | 29.17 | 65.57 |
| Tmax (hr) | | |
| Mean | 6.8 | 6.8 |
| SD | 4.6 | 3.4 |
| Min | 0.0 | 0.0 |
| Median | 8.0 | 8.0 |
| Max | 12.0 | 12.0 |
| N | 14 | 15 |

Table 2. Summarised pharmacokinetic parameters for the different treatments (last treatment day). 1=Minoxidil 5%/Proderm, 2=Regaine 5% topical solution

Statistical and/or Analytical Issues

Values reported below LOQ (0.35 ng/ml) were set to LOQ/2 (0.17 ng/ml) in all pharmacokinetic calculations and in corresponding tables and graphs. Values that were reported as “<ng/ml”, (too small sample volume), were excluded from all calculations. At steady-state, a missing value for the concentration at dosing time, is set by WinNonlin to be the minimum concentration observed in the dosing interval.

Local Tolerance

No subject experienced any local irritation or local itching and no local redness was observed before or after application of the test solution. A few of the subjects displayed local dryness both before and after application of test solution and a few subjects before but not after application. This could be due to a lubricant effect of the study medication. Local dryness was, in the few subjects where it was experienced, mainly observed day 6 and with treatments C and D. As the hair should be washed prior to each application of study medication, the hair was washed twice as often with treatment D (reference Regaine, applied twice daily) as compared to the other treatments. Local folliculitis was observed in some individuals before as well as after application of test solution.

Safety Results and Pulse and Blood Pressure

Pulse, systolic and diastolic blood pressure, day 6 were similar to that on day 1, for treatment 1 and 2, see Tables 3, 4 and 5 respectively.

TABLE 3
Pulse (bpm)

| | |
|---|---|
| Treatment 1 | Treatment 2 |

| | Day 1 | Day 6 | Day 1 | Day 6 |
|---|---|---|---|---|
| Mean | 71 | 67 | 69 | 67 |
| SD | 16 | 11 | 11 | 9 |
| Median | 70 | 67 | 68 | 67 |
| Min | 50 | 49 | 43 | 46 |
| Max | 107 | 93 | 93 | 80 |

TABLE 4
Systolic blood pressure (mm Hg)

| | | Treatment 1 | | Treatment 2 | |
|---|---|---|---|---|---|
| | Pre Study | Day 1 | Day 6 | Day 1 | Day 6 |
| Mean | 123 | 130 | 127 | 130 | 128 |
| SD | 13 | 16 | 14 | 16 | 13 |
| Median | 123 | 127 | 124 | 126 | 126 |
| Min | 105 | 110 | 100 | 100 | 110 |
| Max | 150 | 162 | 149 | 164 | 160 |

TABLE 5
Diastolic blood pressure (mm Hg)

| | | Treatment 1 | | Treatment 2 | |
|---|---|---|---|---|---|
| | Pre Study | Day 1 | Day 6 | Day 1 | Day 6 |
| Mean | 73 | 77 | 79 | 81 | 78 |
| SD | 8 | 12 | 10 | 9 | 12 |
| Median | 75 | 76 | 77 | 80 | 78 |
| Min | 60 | 56 | 55 | 67 | 63 |
| Max | 85 | 97 | 93 | 98 | 100 |

Treatment 1 = Minoxidil 5%/Proderm, once daily
Treatment 2 = Regaine ® 5%, twice daily

Adverse Events

There were 29 adverse events reported, all of which were mild or moderate. Thirteen adverse events were reported during treatment C and sixteen during treatment D. For some subjects the same adverse event was reported twice during the same treatment period. A summary of subjects experiencing at least one occurrence of a specific adverse event is given below (i.e. only one occurrence per subject for each adverse event; n=46).

TABLE 6
Adverse medical events summary¹

Treatment

| Adverse Event | 1 | 2 |
|---|---|---|
| Abdominal distension | | |
| Abdominal pain upper | | |
| Back pain | | |
| Conjunctivitis NEC | 1 | |
| Dry skin | 3 | 6 |
| Erythema NEC | | 1 |
| Folliculitis | 1 | 3 |
| Gout | | |
| Groin pain | | 1 |
| Headache NOS | 1 | 2 |
| Hypertension NOS | | |
| Nasopharyngitis | 1 | 2 |
| Pruritus NOS | 2 | |
| Rhinitis allergic NOS | | |
| Rhinitis NOS | 1 | |
| Scalp tenderness | 1 | |
| Sore throat NOS | | |
| Vasovagal attack | 1 | |

¹Only one occurrence per subject and treatment tabulated
NEC = Not Elsewhere Classified,
NOS = Not Otherwise Specified

The most common symptoms regarded as possible related to the study medications were dry skin, folliculitis, and headache. One subject developed a vasovagal attack in connection to blood sampling, before administration of study medication.

In this study the systemic uptake of minoxidil after once daily application of three different formulations has been compared to the systemic uptake after twice daily appliation of Regaine® 5%. The tested once-daily treatment Proderm had a significantly lower Cₘₐₓ and AUC₀₋₂₄ₕ compared to the twice-daily treatment (Regaine® 5%).

The tested once daily treatment Proderm had significantly lower rate and extent of absorption compared to the twice daily treatment (Regaine® 5%). No differences between the treatments were noticed regarding local tolerance. The Proderm preparation showed less local irritation than the reference preparation of minoxidil.

### Reference List

1. Parfitt K, ed. Martindale. The complete drug reference. 32^{nd} ed. London: Pharmaceutical Press, 1999.

1. A pharmaceutical and/or cosmetical composition, characterised in that it comprises one or more substances active in preventing hair loss and/or stimulating hair growth and a preparation comprising lipophilic and hydrophilic components for application on the skin, which preparation exists as a two-phase system and thereby is capable of creating a semi-permeable membrane in the skin.

2. A composition according to claim 1, characterized in that the active substance is chosen from 2-amino-pyrimidine-4-3-oxides, 2,4-diamino-pyrimidine-3-oxides; 2.4-diamino 6-alkoxy-pyrimidine-3-oxides; 6-substituted 2.4-diamino-pyrimidine-3-oxides; 2,4-diamino-6-alkoxy or 6-thioalkyl-pyrimidine-oxides; 2-alkyl-4-amino (or 2,4-dialkyl or 2.4-diamino)-pyrimidine-3-oxides; 6-haloalkoxy-pyrimidine-3-oxides; 2,4,6-triamino-pyrimidine-3-oxide; 2,4-diamino-6-piperidino-pyrimidine-3-oxide; and derivatives thereof.

3. A composition according to claim 2, characterised in that the active substance is chosen from 6-amino-1,2-dihydro-1-hydroxy-2-iminopyrimidines, their carboxyacylated counterparts, and the corresponding acid addition salts thereof, preferably the one that are substituted in the 4-position and optionally in the 5-position, wherein the substituent in the 4-position being a secondary or tertiary amino moiety analogues and salts thereof.

4. A composition according to claim 3, characterised in that the active compound is 2,4-diamino-6-piperidino-pyrimidine-3-oxide (Minoxidil).

5. A composition according to claim 1, characterised in that the active compound is 17β-N-(monosubstituted)-carbamoyl-4-aza-5.alpha.-androsten-1-en-3 one compounds, especially 17β-(N-tert-butyl carbamoyl)-4-aza-5 alpha-androst-1-en-3-one.

6. A composition according to claim 1, characterised in that the preparation is prepared from lipophilic components and hydrophilic components, the lipophilic components being chosen among fatty acids comprising 14 to 20 carbon atoms and dimethyl polysiloxane and said hydrophilic components being chosen among triethanolamine, monopropylene glycol, glycerol, sorbitol, polyethylene glycol and polyvinyl pyrrolidone.

7. A composition according to claim 1, characterised in that said lipophilic component comprises said fatty acid or any mixture thereof and said hydrophilic component comprises triethanolamine, the molar ratio of fatty acid(mixture thereof):triethanolamine preferably being higher than 1.

8. A composition according to claim 1, characterised in that the molar ratio between fatty acid (mixture thereof):triethanolamine is higher than about 2:1.

9. A composition according to claim 1, characterised in that the fatty acid component is a mixture of fatty acids comprising 14 to 20 carbon atoms, which mixture is of the following composition: at most about 2% of a component comprising 14 carbon atoms, between about 47 and 52% of a component comprising 16 carbon atoms, between about 43 and 48% of a component comprising 18 carbon atoms and at most about 1% of a component comprising 20 carbon atoms.

10. A composition according to claim 1, characterised in that the preparation has been prepared from a fatty acid of 14 to 20 carbon atoms, or any mixture of such fatty acids, triethanolamine, monopropylene glycol, polyvinyl pyrrolidone, dimethyl polysiloxane and a component chosen among glycerol, sorbitol and polyethylene glycol, or a mixture thereof, and water.

11. A composition according to claim 1, characterised in that the preparation has been prepared from about 5 to 8% of a fatty acid comprising 14 to 20 carbon atoms, or a mixture of any such fatty acids, about 0.73 to 2.66% of triethanolamine, about 4.5 to 7.0% of monoproplylene glycol, about 1.5 to 1.97% of polyvinyl pyrrolidone, at most about 5.0% of dimethyl polysiloxane and about 1 to 2% of any one of glycerol, sorbitol or polyethylene glycol, or any mixture thereof, the balance being water up to 100%.

12. (cancelled)

13. A method for treating humans for alopecia, characterised in topically applying the human scalp an effective amount of a composition according to claim 1.
